(19) United States
(12) Patent Application Publication
(10) Pub. No.: US 2021/0127947 A1
(43) Pub. Date: May 6, 2021

(54) MULTI-SECTION BENDING TUBE HAVING GRADUATED RIGIDITY, INSERTION TUBE FOR ENDOSCOPE USING THE BENDING TUBE, AND ENDOSCOPE

(71) Applicant: ANQING MEDICAL CO., LTD, Shanghai (CN)

(72) Inventor: Zhenhua ZHOU, Shanghai (CN)

(21) Appl. No.: 16/753,934

(22) PCT Filed: Oct. 20, 2017
(86) PCT No.: PCT/CN2017/106956

§ 371 Date: Apr. 6, 2020

(51) Int. Cl. A61B 1/008; A61B 1/01; A61B 1/005

(52) U.S. Cl. CPC A61B 1/008; A61B 1/0052; A61B 1/01

(57) ABSTRACT

The present invention provides a multi-section bending tube (100) having graduated rigidity, applied to an insertion portion of an endoscope (300). The multi-section bending tube (100) having graduated rigidity sequentially includes, from a non-insertion end to an insertion end: a straight catheter portion (1), a transition catheter portion (2) and a snake bone catheter portion (3), which have equal inside diameters and are connected in sequence, wherein the straight catheter portion (1) is a cylindrical catheter with a fully-enclosed catheter wall, helical slits (21) for providing a bending space of a tube body are disposed on the catheter wall of the transition catheter portion (2), the snake bone catheter portion (3) includes a plurality of tube joints (31) connected with each other in series, and the tube joints (31) are connected with each other in a clamping or hinging manner. The multi-section bending tube (100) having graduated rigidity is capable of increasing the coaxial steering proportion of an endoscope insertion tube (200) and greatly reducing the coaxial error of the endoscope insertion tube (200), thereby reducing the autogenous twisting of a laser optical fiber, prolonging the service life of the instrument, reducing the friction coefficient between the endoscope body and a ureter sheath, reducing the operation difficulty and having a high practical value.

### FIELD OF THE INVENTION

The present invention relates to the field of medical equipment, and in particular to a multi-section bending tube having graduated rigidity, an insertion tube for an endoscope using the bending tube, and the endoscope.

### BACKGROUND OF THE INVENTION

With the development of science and technology, an endoscope has been widely used in the medical field, and it is one of the important tools for examining internal organs of the human body. Since its discovery in 1806, the development of the endoscope has gone through the following four stages: a rigid tube endoscope, a semi-flexible endoscope, a fiber endoscope, an ultrasonic and electronic endoscope and the like. At present, the types of endoscopes are mainly divided into rigid tube endoscopes and hose endoscopes. The rigid tube endoscope has high strength and good insertability, but it is easy to puncture an inner wall, and because the lens cannot be rotated, the rigid tube endoscope can only be used for performing some operations with relatively clear lesion locations. For some operations where the lesion locations are unclear, it is necessary to constantly adjust the direction for observation. Therefore, a soft endoscope needs to be adopted. However, since the soft endoscope is made of a relatively soft material, it is difficult to provide sufficient insertability and radial expandability for an insertion portion of the endoscope. In order to combine the advantages of the two endoscopes, a variety of improved endoscopes have now appeared.

For example, the Chinese patent document with the application number of CN201510770248.0 provides an endoscope bending tube, which is simple in structure, convenient to process and convenient to install members. The technical solution adopted by the present invention is as follows: the endoscope bending tube includes two semicircular tube-shaped bendable composition portions, the two composition portions form a tube body with an inner tube cavity after being docked, a plurality of compression grooves that are distributed in the radial direction and used for providing a bending space for the tube body are arranged on the axial direction of the composition portions, and the radian of the plurality of compression grooves is less than that of the composition portions; in the endoscope bending tube of the present invention, the two composition portions are connected to form the tube body of the bending tube, during the processing, the compression grooves can be quickly formed in each composition portion, meanwhile, guide holes for enabling bending guide lines to pass through can be formed in the composition portions in advance, and the bending guide lines are placed in the guide holes, the members of the endoscope are disposed on the corresponding composition portions, finally the two composition portions are connected, the two connected composition portions form the endoscope bending tube together, the endoscope bending tube can be bent toward two directions provided with the compression grooves through the control of the bending guide lines to complete the functions of the endoscope bending tube, thereby realizing the processing and mounting of the endoscope bending tube quickly, in this way, the production efficiency can be greatly improved, and the mounting labor intensity of workers is reduced; and meanwhile, the structure is simple, the processing is extremely convenient, the manufacturing cost is low, meanwhile the processing precision can be better controlled, and the production quality is improved.

The endoscope provided by the Chinese patent document with the application number of CN201510946003.9 adopts a novel bending tube, the bending tube includes a tube body and guide line bodies, the tube body and the guide line bodies are integrally formed by a resin material, the tube body is provided with a plurality of notches, and the notches divide the tube body into a plurality of unit joints and connecting portions connected between the adjacent unit joints. The guide line bodies are fixed on the inner wall of the tube body and form a channel for enabling a linear transmission member to pass through, the guide line bodies are divided into at least one group, and each guide line body group includes a plurality of guide line bodies. The bending tube has a simple structure, and the bending of the bending tube is mainly achieved by the own characteristics of the resin material in cooperation with the notches on the tube body under the pull of the linear transmission member. The tube body and the guide line bodies are integrally formed by the resin material, and the unit joints of the bending tube are connected by the own connection portions, so no complicated connection structure is needed, and the cost is greatly reduced. At the same time, the requirements of low-cost disposable endoscopes can also be met.

The rotation coaxiality between the non-insertion end and the insertion end of the bending tube in the prior art is very different, sometimes is up to 30 degrees, and the rotation coaxial error at the both ends of the bending tube is very large, which leads to relatively large autogenous twisting of the laser optical fiber, so that the service life of the instrument is shortened, furthermore, the friction coefficient between the endoscope body and the ureter sheath is increased, making it difficult to use the endoscope to enter the human cavity, and reducing the surgical efficiency

### SUMMARY OF THE INVENTION

The present invention discloses a multi-section bending tube having graduated rigidity, applied to an insertion portion of an endoscope, wherein the multi-section bending tube having graduated rigidity sequentially includes, from a non-insertion end to an insertion end: a straight catheter portion, a transition catheter portion and a snake bone catheter portion, which have equal inside diameters and are connected in sequence, the straight catheter portion is a cylindrical catheter with a fully-enclosed catheter wall, slits for providing a bending space of a tube body are disposed on the catheter wall of the transition catheter portion, the snake bone catheter portion includes a plurality of tube joints connected with each other in series, the tube joints are connected with each other in a clamping or hinging manner, and the curvature of the transition catheter portion is greater than the curvature of the straight catheter portion and is less than the curvature of the snake bone catheter portion.

Further, helical slits are disposed on the catheter wall of the transition catheter portion, and the screw pitches among the helical slits of the transition catheter portion are equal.

Preferably, the transition catheter portion and the snake bone catheter portion are connected by an elongated tube joint.

Preferably, the snake bone catheter portion includes a grouped tube joint portion and an isometric tube joint portion from the non-insertion end to the insertion end, the grouped tube joint portion includes multiple groups of snake bone catheters, and each group of snake bone catheters is composed of a plurality of tube joints with the same length and connected in series, the groups of snake bone catheters are connected by elongated tube joints, and the numbers of tube joints of the groups of snake bone catheters gradually increase from the non-insertion end to the insertion end.

Further, the axial lengths of the plurality of tube joints of the snake bone catheter portion gradually decrease from the non-insertion end to the insertion end.

Preferably, any tube joint is provided with a convex portion that can be clamped in a corresponding concave portion of the adjacent tube joint.

Preferably, a seam with a certain width is disposed at the connection site of the tube joints on both sides of the convex portion of the snake bone catheter portion.

Preferably, one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

The present invention further discloses an insertion tube for an endoscope, including: the multi-section bending tube having graduated rigidity as described above; and a protective sheath covering the multi-section bending tube having graduated rigidity.

The present invention further discloses an endoscope, including the insertion tube for the endoscope as described above, a control device and a handle; one or more traction wires are disposed in the insertion tube, one end of the traction wire is fixedly connected with the insertion end of the insertion tube, and the other end of the traction wire is connected with the control device disposed on the handle for controlling the bending of the insertion tube.

In summary, according to the multi-section bending tube having graduated rigidity, the insertion tube for the endoscope using the bending tube, and the endoscope involved in the present invention, the multi-section bending tube having graduated rigidity is formed by sequentially connecting three-section catheters with different structures, compared with the traditional endoscope insertion tube, the coaxial steering proportion of the endoscope insertion tube can be improved, so that the axial ratio of the insertion end to the non-insertion end is maximally close to 1:1, and the coaxial error of the endoscope insertion tube is greatly reduced, so that the autogenous twisting of the laser optical fiber can be reduced, the service life of the instrument is prolonged, furthermore, the friction coefficient between the endoscope body and a ureter sheath is reduced, the operation difficulty is reduced, and the practical value is very high.

In order to make the above contents of the present invention more comprehensible, preferred embodiments are listed below and are described in detail below in combination with the drawings.

### BRIEF DESCRIPTION OF THE DRAWINGS

The present invention is described below with reference to the drawings.

FIG. 1 is a schematic structural diagram of one side face of a multi-section bending tube having graduated rigidity in a first embodiment of the present invention;

FIG. 2 is a schematic structural diagram of another side face of the multi-section bending tube having graduated rigidity in the first embodiment of the present invention;

FIG. 3 is a schematic structural diagram of a tube joint of a snake bone catheter portion of the multi-section bending tube having graduated rigidity in the first embodiment of the present invention;

FIG. 4 is a sectional view of the multi-section bending tube having graduated rigidity in the first embodiment of the present invention;

FIG. 5-1 is a schematic structural diagram of a tube joint of a snake bone catheter portion of a multi-section bending tube having graduated rigidity in a third embodiment of the present invention;

FIG. 5-2 is another schematic structural diagram of the tube joint of the snake bone catheter portion of the multi-section bending tube having graduated rigidity in the third embodiment of the present invention;

FIG. 6 is a schematic diagram of an overall structure of an endoscope using a multi-section bending tube having graduated rigidity in a fifth embodiment of the present invention.

### DETAILED DESCRIPTION OF THE EMBODIMENTS

The implementation manner of the present invention is illustrated by specific embodiments below. Those skilled in the art can easily understand other advantages and effects of the present invention from the contents disclosed in the specification.

Exemplary embodiments of the present invention will now be described with reference to the drawings, however, the present invention can be implemented in many different forms and is not limited to the embodiments described herein, and these embodiments are provided to completely disclose the present invention in detail and to fully convey the scope of the present invention to those skilled in the art. The terms in the exemplary embodiments shown in the drawings are not limitations to the present invention. In the drawings, the same units/elements use the same reference signs.

Unless otherwise stated, the terms (including scientific and technical terms) used herein have the ordinary meanings to those skilled in the art to which the present invention belongs. In addition, it can be understood that the terms defined in commonly used dictionaries should be understood as having meanings consistent with the context of their related fields, and should not be understood as idealized or over formal meanings.

### First Embodiment

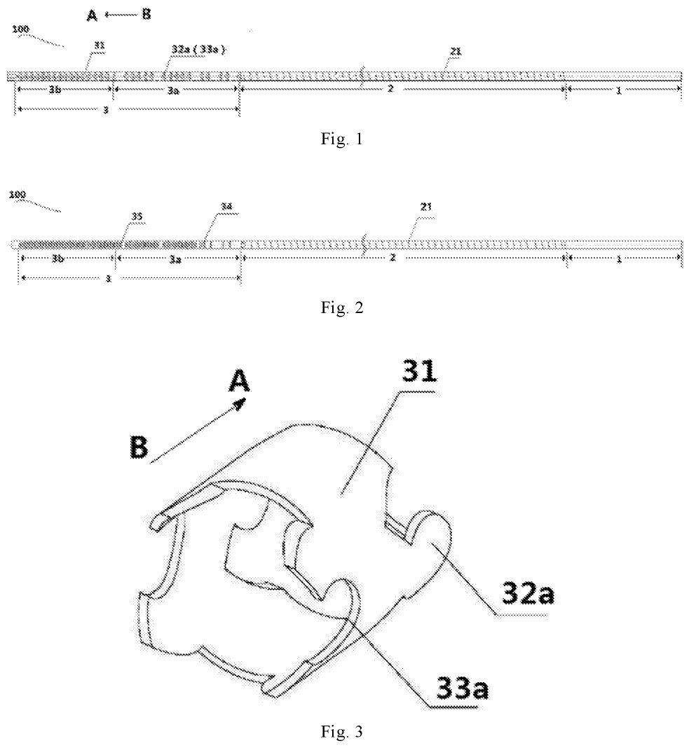

As shown in FIG. 1, the first embodiment of the present invention discloses a multi-section bending tube 100 having graduated rigidity, applied to an insertion portion of an endoscope, wherein the multi-section bending tube 100 having graduated rigidity sequentially includes, from a non-insertion end to an insertion end: a straight catheter portion 1, a transition catheter portion 2 and a snake bone catheter portion 3, which have equal inside diameters and are connected in sequence, the straight catheter portion 1 is a cylindrical catheter with a fully-enclosed catheter wall, helical slits 21 for providing a bending space of a tube body are disposed on the catheter wall of the transition catheter portion 2, the snake bone catheter portion 3 includes a plurality of tube joints 31 connected with each other in series, and any tube joint 31 is provided with a first convex portion 32a that can be clamped in a corresponding first concave portion 33a of the adjacent tube joint.

Preferably, the slits of the transition catheter portion 2 in the present embodiment are helical slits or netty slits. As shown in FIG. 2, the screw pitches between the helical slits 21 are equal. The equal screw pitch design can make all parts of the transition catheter part have the same twistability, which is convenient for the control of an operator. Further, the screw pitches between the helical slits 21 can also change from large to small or from small to large, so that the hardness of the part of catheter gradually changes along the axial direction to meet the needs of more occasions.

In the present embodiment, as shown in FIG. 1, the transition catheter portion 2 and the snake bone catheter portion 3 are connected by an elongated tube joint in which the axial length is equal to the axial length of one or two tube joint, the snake bone catheter portion 3 includes a grouped tube joint portion 3a and an isometric tube joint portion 3b from the non-insertion end to the insertion end, and the isometric tube joint portion 3b is composed of m standard tube joints that have the same axial length and are connected in series with each other; the grouped tube joint portion 3a includes multiple groups of snake bone catheters and a plurality of elongated tube joints, each group of snake bone catheters is composed of n standard tube joints that have the same axial length and are connected in series with each other, and the length of the elongated tube joint is greater than the axial length of one standard tube joint and is less than the axial length of four standard tube joints. The groups of snake bone catheters are connected by the elongated tube joints, the grouped tube joint portion 3a and the isometric tube joint portion 3b are connected by the elongated tube joints, and the grouped tube joint portion 3a and the transition catheter portion 2 are connected by the elongated tube joints. N and m are both positive integers, preferably n is less than or equal to 5, and m is a reasonable number greater than or equal to 10. In the present embodiment, the grouped tube joint portion 3a includes 4 groups of snake bone catheters, wherein the numbers of tube joints of the groups of snake bone catheters gradually increase from the non-insertion end to the insertion end, as shown in FIG. 1, the numbers of tube joints of the groups of snake bone catheters are respectively 1, 1, 4, 4 in order; and the grouped tube joint portion 3a at the front end of a grouped catheter portion 21 is composed of about 15 standard tube joints that have the same axial length and are connected in series with each other. Therefore, the multi-section bending tube having graduated rigidity of the present invention forms a front end with good flexibility, and the snake bone catheter portion 3 can be bent into a closed circular ring shape. After being bent, the deflection angle between the axis of a top end face of the snake bone catheter portion and the axis of the snake bone catheter portion before bending can exceed 270 degrees. It can be seen from FIG. 1 that due to the above change in the lengths of the tube joints of the snake bone catheter portion 3 of the multi-section bending tube having graduated rigidity in the present embodiment, the rigidity gradually decreases from the non-insertion end to the insertion end, so the isometric tube joint portion 3b has good soft bending property, and good guidance is provided for the insertion of the multi-section bending tube having graduated rigidity; the grouped tube joint portion 3a has high hardness, and thus can play a good supporting role when the isometric tube joint portion 3b is bent. The setting of the two kinds of tube joints is not limited to the number provided in the present embodiment, and can be designed in more detail according to actual needs.

In the present embodiment, the rotation coaxiality of the straight catheter portion 1 is 100%, that is, the rotation axis of the straight catheter portion is always straight, and when angular deflection occurs at one end, the same angular deflection also occurs at the other end; the helical slits 21 are disposed on the catheter wall of the transition catheter portion 2, the helical slits 21 can provide a space for the bending of the tube body, so that the transition catheter portion can be bent, when the angular deflection occurs at one end, the same angular deflection also occurs at the other end, since the bending space provided by the slits 21 of the transition catheter section 2 is limited, the rotation coaxiality of this part of catheter is slightly lower than that of the straight catheter section 1, and the widths of the slits can be designed to meet the actual needs; the snake bone catheter portion 3 connected in series to the insertion end in front of the transition catheter section 2 includes a plurality of tube joints 31 connected with each other in series, the tube joints 31 are connected with each other in a clamping or hinging manner, and the snake bone catheter portion composed of the plurality of tube joints that are connected with each other in series can have greater twistability. When the straight catheter portion 1, the transition catheter portion 2 and the snake bone catheter portion 3 are sequentially connected, the rigidity of the multi-section bending tube having graduated rigidity in the present embodiment gradually decreases from the non-insertion end to the insertion end, the twistability is gradually enhanced, compared with the traditional endoscope bending tube of a single structure, the multi-section bending tube having graduated rigidity has better controllability, can not only bend forward according to the internal cavity of the human body, but also can make the coaxial ratio of the insertion end to the non-insertion end maximally close to 1:1, and greatly reduce the coaxial error of the endoscope insertion tube, therefore the autogenous twisting of a laser optical fiber can be reduced, the service life of the instrument is prolonged, furthermore, the friction coefficient between the endoscope body and a ureter sheath is reduced, the operation difficulty is reduced, and the practical value is very high.

Preferably, as shown in FIG. 1 and FIG. 3, any tube joint 31 is provided with a first convex portion 32a that can be clamped in a corresponding first concave portion 33a of the adjacent tube joint, the first convex portion 32a and the first concave portion 33a both extend along the axial direction of the tube body, the first convex portion 32a and the first concave portion 33a cooperate with each other, the width of the front end of the first convex portion 32a is greater than the width of the tail end thereof (as shown by the arrow direction in FIG. 1, A represents the front end, and B represents the tail end), correspondingly, the width of the front end of the first concave portion 33a is greater than the width of the tail end thereof, so that the first convex portion 32a and the first concave portion 33a are clamped with each other. When the controllable bending tube is stretched along the axial direction, since the width of the front end of the first convex portion 32a is greater, the first convex portion cannot pass through the narrower rear end of the first concave portion 33a, so that the respective tube joints 31 are clamped in the axial direction without being excessively stretched, and at the same time, the excessive twisting of the multi-section bending tube having graduated rigidity in the present invention in the radial direction is further avoided.

Preferably, in the present embodiment, the length of the straight catheter portion can be 5.5% to 6% of that of the multi-section bending tube having graduated rigidity, the length of the transition catheter portion is 82% to 83.5% of that of the multi-section bending tube having graduated rigidity, the length of the snake bone catheter portion is 11% to 12% of that of the multi-section bending tube having graduated rigidity, wherein the length of the grouped tube joint portion is approximately 45% of the length of the snake bone catheter portion. The multi-section bending tube having graduated rigidity meeting the above-mentioned length ratio range can obtain the optimal rotation coaxiality. Those skilled in the art can think of that the length ratios of various parts can also be designed according to the actual needs.

Preferably, as shown in FIG. 2, a seam 34 with a certain width is disposed at the connection site of the tube joints on both sides of the first convex portion 32a of the snake bone catheter portion 3, and the seam 34 can provide the bending space of the tube body, so that the tube body is bent toward the direction where a through hole is located.

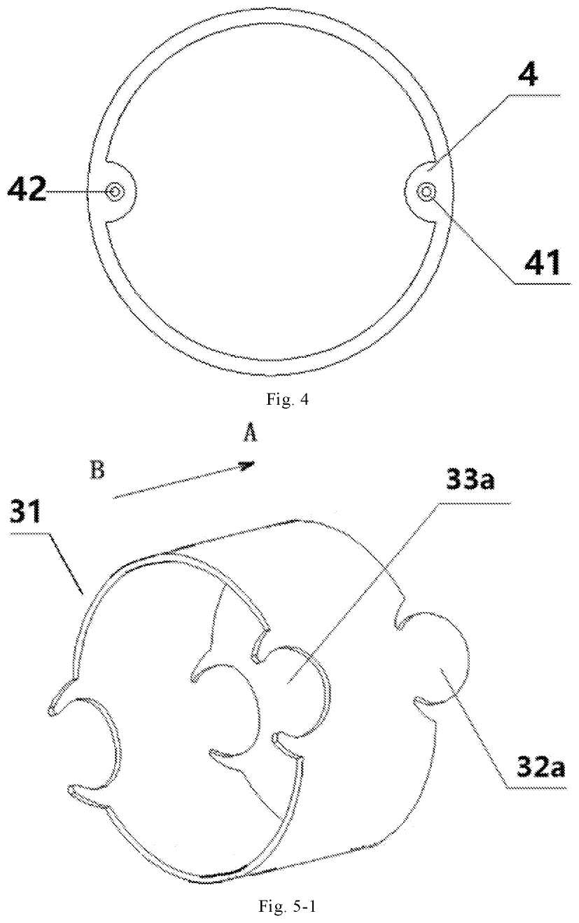

More preferably, as shown in FIG. 4, one or more convex ribs 4 are formed on an inner wall of the multi-section bending tube having graduated rigidity, a traction hole 41 is further formed in each convex rib 4, the traction hole 41 can be used for accommodating a traction wire 42, and the bending of the multi-section bending tube having graduated rigidity can be controlled by controlling the traction wire 42. One or more convex ribs 4 extending from one end of the tube to the other end of the tube are formed on the inner tube wall of the multi-section bending tube having graduated rigidity, the traction hole 41 is further formed in each convex rib 4, the shape of the traction hole 41 is not particularly limited, as long as the traction hole 41 can be used for accommodating the traction wire 42, for example, the traction hole can be circular or oval, and the bending of the multi-section bending tube having graduated rigidity can be controlled by controlling the traction wire 42. When the multi-section bending tube having graduated rigidity disclosed in the present invention is manufactured, a tube with the convex rib 4 and the traction hole 41 in the convex rib 4 can be directly prepared by an extrusion die and other methods, and the tube with the convex rib 4 and the traction hole 41 is integrally formed directly. Thereafter, the tube becomes a bendable tube through common cutting methods in the industry.

Further, the number of the convex ribs 4 is two, and the tube can be controlled to bend in two directions when the traction wire 42 is inserted; and more preferably, the number of the convex ribs 4 is four, and the tube can be bent toward multiple angles through the cooperation of traction forces in multiple directions.

Further, the center line of the tube is taken as an axis in the multi-section bending tube having graduated rigidity, and preferably, the convex ribs 4 are symmetrically disposed in the controllable bending tube. The number of the convex ribs 4 corresponds to the number of the traction wires 42 inserted therein. When there are two convex ribs 4, that is, two traction holes 41 are formed for enabling the traction wires 42 to pass through, two corresponding traction wire 42 are also provided, the insertion end of the endoscope can be controlled to bend in two directions where the traction wires 42 are located, and it can be ensured that various angles are observed by rotating an endoscope insertion tube 100; and when there are four convex ribs 4, the insertion end of the endoscope can be controlled to bend in any direction, and any angle in the cavity can also be observed without rotating the endoscope.

Further, the multi-section bending tube having graduated rigidity is made of a metal material or plastic. The non-metal material can be selected from various plastic materials such as nylon, polyurethane (PU), thermoplastic polyurethane elastomer rubber (TPU), polyvinyl chloride (PVC), thermoplastic elastomer (TPE), etc.

Further, in the present preferred embodiment, the traction wire 42 is preferably a steel wire, but the present invention is not limited thereto, the traction wire 42 can also be any filament-like structure, and the filament-like structure can also be selected from any metal or non-metal material.

### Second Embodiment

The second embodiment of the present invention discloses a multi-section bending tube 100 having graduated rigidity, applied to an insertion portion of an endoscope, wherein the multi-section bending tube 100 having graduated rigidity sequentially includes, from a non-insertion end to an insertion end: a straight catheter portion 1, a transition catheter portion 2 and a snake bone catheter portion 3, which have equal inside diameters and are connected in sequence, the straight catheter portion 1 is a cylindrical catheter with a fully-enclosed catheter wall, slits 21 for providing a bending space of a tube body are disposed on the catheter wall of the transition catheter portion 2, the snake bone catheter portion 3 includes a plurality of tube joints 31 connected with each other in series, the tube joints 31 are connected with each other in a clamping or hinging manner, and the curvature of the transition catheter portion is greater than the curvature of the straight catheter portion and is less than the curvature of the snake bone catheter portion.

The present embodiment is a modification of the first embodiment, the difference between the present embodiment and the first embodiment lies in that the axial lengths of the plurality of tube joints 31 of the snake bone catheter portion 3 gradually decrease from the non-insertion end to the insertion end, therefore, the insertion end has good soft bending property, and good guidance is provided for the insertion of the multi-section bending tube having graduated rigidity; and the non-insertion end has higher hardness, and can be used as a transition part of the transition catheter portion 2 and the snake bone catheter portion 3 to provide good guidance for the insertion of the multi-section bending tube having graduated rigidity, so that the technical effect is very excellent.

Preferably, any tube joint 31 is provided with a first convex portion 32a that can be clamped in a corresponding first concave portion 33a of the adjacent tube joint.

As shown in FIG. 4, one or more convex ribs 4 are formed on an inner wall of the multi-section bending tube having graduated rigidity, a traction hole 41 is further formed in each convex rib 4, the traction hole 41 can be used for accommodating a traction wire 42, and the bending of the multi-section bending tube having graduated rigidity can be controlled by controlling the traction wire 42. One or more convex ribs 4 extending from one end of the tube to the other end of the tube are formed on the inner tube wall of the multi-section bending tube having graduated rigidity, the traction hole 41 is further formed in each convex rib 4, the shape of the traction hole 41 is not particularly limited, as long as the traction hole 41 can be used for accommodating the traction wire 42, for example, the traction hole can be circular or oval, and the bending of the multi-section bending tube having graduated rigidity can be controlled by controlling the traction wire 42. When the multi-section bending tube having graduated rigidity disclosed in the present invention is manufactured, a tube with the convex rib 4 and the traction hole 41 in the convex rib 4 can be directly prepared by an extrusion die and other methods, and the tube with the convex rib 4 and the traction hole 41 is integrally formed directly. Thereafter, the tube becomes a bendable tube through common cutting methods in the industry.

### Third Embodiment

The third embodiment of the present invention discloses a multi-section bending tube having graduated rigidity, applied to an insertion portion of an endoscope, wherein the multi-section bending tube having graduated rigidity sequentially includes, from a non-insertion end to an insertion end: a straight catheter portion 1, a transition catheter portion 2 and a snake bone catheter portion 3, which have equal inside diameters and are connected in sequence, the straight catheter portion 1 is a cylindrical catheter with a fully-enclosed catheter wall, helical slits 21 for providing a bending space of a tube body are disposed on the catheter wall of the transition catheter portion 2, the snake bone catheter portion 3 includes a plurality of tube joints 31 connected with each other in series, the tube joints are connected with each other in a clamping or hinging manner, and the curvature of the transition catheter portion is greater than the curvature of the straight catheter portion and is less than the curvature of the snake bone catheter portion.

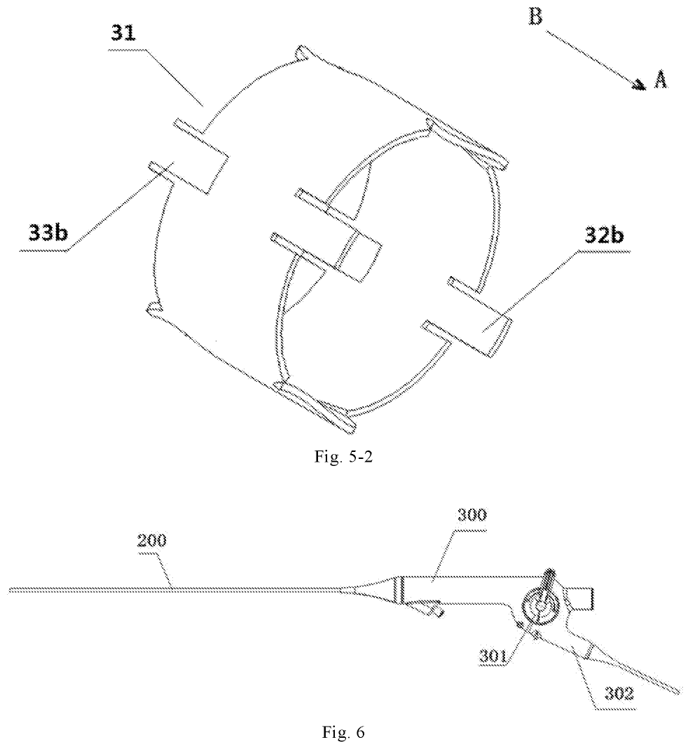

Preferably, any tube joint 31 is provided with a first convex portion 32a that can be clamped in a corresponding first concave portion 33a of the adjacent tube joint. The present embodiment is a modification of the first embodiment, the difference between the present embodiment and the first embodiment lies in that, as shown in FIG. 2, FIG. 5-1 and FIG. 5-2, a seam 34 with a certain width is disposed at the connection site of the tube joints on both sides of the first convex portion 32a of the snake bone catheter portion 3, a second convex portion 32b extending along the axial direction of the circular tube is disposed on one side of all or a part of seams 34, a second concave portion 33b extending along the axial direction of the circular tube is disposed at a position corresponding to the first convex portion 32a on the other side, the second convex portion 32b and the second concave portion 33b cooperate with each other, the width of the front end of the first convex portion 32a is less than the width of the tail end thereof (as shown by the arrow direction in FIG. 3, A represents the front end, and B represents the tail end), correspondingly, the width of the front end of the first concave portion 33a is less than the width of the tail end thereof, so that the second convex portion 32b can reciprocate in the second concave portion 33b along the axial direction of the circular tube.

Specifically, the second convex portion 32b can be rectangular, square, trapezoidal or triangular, and the second concave portion 33b can also be rectangular, square, trapezoidal or triangular. When the second convex portion and the second concave portion are both correspondingly rectangular, square, trapezoidal or triangular, when the snake bone catheter portion 3 is twisted along the radial direction thereof, a twisting space reserved between the second convex portion 32b and the second concave portion 33b is relatively small, such that the twisting angle of the snake bone catheter portion 3 is relatively small; and when the second convex portion 32b is trapezoidal or triangular and the second concave portion 33b is rectangular or square, when the snake bone catheter portion 3 is twisted along the radial direction thereof the twisting space reserved between the second convex portion 32b and the second concave portion 33b is relatively large, such that the twisting angle of the snake bone catheter portion 3 is relatively large. Those skilled in the art can think that the second convex portion 32b and the second concave portion 33b can also have other shapes playing the same role of preventing the twisting, and are not limited to those disclosed in the present embodiment.

In the present embodiment, through the design of the second convex portion and the second concave portion which can reciprocate relative to each other, the twisting of the multi-section bending tube having graduated rigidity can be limited in the radial direction, thereby preventing the excursion of an insertion position or the twisting of the traction wire 42 due to the excessive twisting of the pipeline, so that the operation is more convenient.

### Fourth Embodiment

The present embodiment discloses an insertion tube for an endoscope, including:

a multi-section bending tube having graduated rigidity, and a protective sheath covering the multi-section bending tube having graduated rigidity, the multi-section bending tube having graduated rigidity sequentially includes, from a non-insertion end to an insertion end: a straight catheter portion 1, a transition catheter portion 2 and a snake bone catheter portion 3, which have equal inside diameters and are connected in sequence, the straight catheter portion 1 is a cylindrical catheter with a fully-enclosed catheter wall, helical slits 21 for providing a bending space of a tube body are disposed on the catheter wall of the transition catheter portion 2, the tube joints are connected with each other in a clamping or hinging manner, and the curvature of the transition catheter portion is greater than the curvature of the straight catheter portion and is less than the curvature of the snake bone catheter portion.

In the present embodiment, any tube joint 31 is provided with a first convex portion 32a that can be clamped in a corresponding first concave portion 33a of the adjacent tube joint. Preferably, as shown in FIG. 2, the screw pitches between the helical slits 21 of the transition catheter portion 2 in the present embodiment are equal. The equal screw pitch design can make all parts of the transition catheter part have the same twistability, which is convenient for the control of the operator. Further, the screw pitches between the helical slits 21 can also change from large to small or from small to large, so that the hardness of the part of catheter gradually changes along the axial direction to meet the needs of more occasions.

In the present embodiment, as shown in FIG. 1, the transition catheter portion 2 and the snake bone catheter portion 3 are connected by an elongated tube joint in which the axial length is equal to the axial length of one or two tube joint, the snake bone catheter portion 3 includes a grouped tube joint portion 3a and an isometric tube joint portion 3b from the non-insertion end to the insertion end, and the isometric tube joint portion 3b is composed of m standard tube joints that have the same axial length and are connected in series with each other; the grouped tube joint portion 3a includes multiple groups of snake bone catheters and a plurality of elongated tube joints, each group of snake bone catheters is composed of n standard tube joints that have the same axial length and are connected in series with each other, and the length of the elongated tube joint is greater than the axial length of one standard tube joint and is less than the axial length of four standard tube joints. The groups of snake bone catheters are connected by the elongated tube joints, the grouped tube joint portion 3a and the isometric tube joint portion 3b are connected by the elongated tube joints, and the grouped tube joint portion 3a and the transition catheter portion 2 are connected by the elongated tube joints. N and m are both positive integers, preferably n is less than or equal to 5, and m is a reasonable number greater than or equal to 10. In the present embodiment, the grouped tube joint portion 3a includes 4 groups of snake bone catheters, wherein the numbers of tube joints of the groups of snake bone catheters gradually increase from the non-insertion end to the insertion end, as shown in FIG. 1, the numbers of tube joints of the groups of snake bone catheters are respectively 1, 1, 4, 4 in order; and the grouped tube joint portion 3a at the front end of the grouped catheter portion 21 is composed of about 15 standard tube joints that have the same axial length and are connected in series with each other. Therefore, the multi-section bending tube having graduated rigidity of the present invention forms a front end with good flexibility, and the snake bone catheter portion 3 can be bent into a closed circular ring shape. After being bent, the deflection angle between the axis of a top end face of the snake bone catheter portion and the axis of the snake bone catheter portion before bending can exceed 270 degrees. Specifically, the rotation coaxiality of the straight catheter portion 1 is 100%, that is, the rotation axis of the straight catheter portion is always straight, and when angular deflection occurs at one end, the same angular deflection also occurs at the other end; the helical slits 21 are disposed on the catheter wall of the transition catheter portion 2, the helical slits 21 can provide a space for the bending of the tube body, so that the transition catheter portion can be bent, when the angular deflection occurs at one end, the same angular deflection also occurs at the other end, since the bending space provided by the slits 21 of the transition catheter section 2 is limited, the rotation coaxiality of this part of catheter is slightly lower than that of the straight catheter section 1, and the widths of the slits can be designed to meet the actual needs; the snake bone catheter portion 3 connected in series to the insertion end in front of the transition catheter section 2 includes a plurality of tube joints 31 connected with each other in series, the tube joints 31 are connected with each other in the clamping or hinging manner, and the snake bone catheter portion composed of the plurality of tube joints that are connected with each other in series can have greater twistability. When the straight catheter portion 1, the transition catheter portion 2 and the snake bone catheter portion 3 are sequentially connected, the rigidity of the multi-section bending tube 100 having graduated rigidity in the present embodiment gradually decreases from the non-insertion end to the insertion end, the twistability is gradually enhanced, compared with the traditional endoscope bending tube of a single structure, the multi-section bending tube having graduated rigidity has better controllability, can not only bend forward according to the internal cavity of the human body, but also can make the coaxial ratio of the insertion end to the non-insertion end maximally close to 1:1, and greatly reduce the coaxial error of the endoscope insertion tube, therefore the autogenous twisting of the laser optical fiber can be reduced, the service life of the instrument is prolonged, furthermore, the friction coefficient between the endoscope body and the ureter sheath is reduced, the operation difficulty is reduced, and the practical value is very high.

Further, the protective sheath is preferably made of polyvinyl chloride, polyurethane, block polyetheramide elastomer or polyamide. The polyvinyl chloride material has common biocompatibility, slightly better strength, but has relatively high hardness and worse bendability, and thus cannot be bent according to the cavity demands under a human body temperature; the polyamide has better biocompatibility, but has greater hydroscopicity, so that the size stability is affected to a certain extent in the complicated internal environment of the human body, and the biocompatibility and strength thereof are slightly worse than those of polyurethane. However, these two materials are low in cost and widely used in industry, and can also meet the use needs of the insertion end of the endoscope.

Compared with other biocompatible materials, the polyurethane and the block polyetheramide elastomer are more preferably selected as the protective sheath, the polyurethane has a good twistability while meeting the human body temperature, ca be bent according to the cavity condition without hurting the body tissues, furthermore, the polyurethane is non-toxic, has good compatibility with the body tissues, and fully guarantees the bendability of the insertion tube 100 at the same time; and the block polyetheramide elastomer (PEBAX) is a thermoplastic elastomer containing no plasticizer, which has good biocompatibility, is extremely suitable for human body, and also has a relatively wide hardness range and good resilience; polyamide products have significant processing properties and biocompatibility, and have excellent softness/flexibility (wide range, and good hand feeling and touch feeling), can provide good guidance and insertion strength for the insertion end; the block polyetheramide elastomer has good stability, few changes in properties, high corrosion resistance and ageing property, and is not hardened at a low temperature, and the structure of the endoscope prepared accordingly does not change even after long-term placement.

### Fifth Embodiment

As shown in FIG. 6, the fifth embodiment of the present invention discloses an endoscope 300, including an insertion tube 200 for the endoscope, a control device 301 and a handle 302; one or more traction wires 42 are disposed in the insertion tube, one end of the traction wire 42 is fixedly connected with the insertion end of the insertion tube, and the other end of the traction wire is connected with the control device 301 disposed on the handle 302 for controlling the bending of the insertion tube; and

the insertion tube 200 for the endoscope includes: a multi-section bending tube having graduated rigidity, and a protective sheath covering the multi-section bending tube having graduated rigidity, the multi-section bending tube having graduated rigidity sequentially includes, from a non-insertion end to an insertion end: a straight catheter portion 1, a transition catheter portion 2 and a snake bone catheter portion 3, which have equal inside diameters and are connected in sequence, the straight catheter portion 1 is a cylindrical catheter with a fully-enclosed catheter wall, helical slits 21 for providing a bending space of a tube body are disposed on the catheter wall of the transition catheter portion 2, the snake bone catheter portion 3 includes a plurality of tube joints 31 connected with each other in series, the tube joints are connected with each other in a clamping or hinging manner, and the curvature of the transition catheter portion is greater than the curvature of the straight catheter portion and is less than the curvature of the snake bone catheter portion.

Preferably, as shown in FIG. 2, the screw pitches between the helical slits 21 of the transition catheter portion 2 in the present embodiment are equal. The equal screw pitch design can make all parts of the transition catheter part have the same twistability, which is convenient for the control of the operator. Further, the screw pitches between the helical slits 21 can also change from large to small or from small to large, so that the hardness of the part of catheter gradually changes along the axial direction to meet the needs of more occasions.

In the present embodiment, as shown in FIG. 1, the transition catheter portion 2 and the snake bone catheter portion 3 are connected by an elongated tube joint in which the axial length is equal to the axial length of one or two tube joint, the snake bone catheter portion 3 includes a grouped tube joint portion 3a and an isometric tube joint portion 3b from the non-insertion end to the insertion end, and the isometric tube joint portion 3b is composed of m standard tube joints that have the same axial length and are connected in series with each other; the grouped tube joint portion 3a includes multiple groups of snake bone catheters and a plurality of elongated tube joints, each group of snake bone catheters is composed of n standard tube joints that have the same axial length and are connected in series with each other, and the length of the elongated tube joint is greater than the axial length of one standard tube joint and is less than the axial length of four standard tube joints. The groups of snake bone catheters are connected by the elongated tube joints, the grouped tube joint portion 3a and the isometric tube joint portion 3b are connected by the elongated tube joints, and the grouped tube joint portion 3a and the transition catheter portion 2 are connected by the elongated tube joints. N and m are both positive integers, preferably n is less than or equal to 5, and m is a reasonable number greater than or equal to 10. In the present embodiment, the grouped tube joint portion 3a includes 4 groups of snake bone catheters, wherein the numbers of tube joints of the groups of snake bone catheters gradually increase from the non-insertion end to the insertion end, as shown in FIG. 1, the numbers of tube joints of the groups of snake bone catheters are respectively 1, 1, 4, 4 in order; and the grouped tube joint portion 3a at the front end of the grouped catheter portion 21 is composed of about 15 standard tube joints that have the same axial length and are connected in series with each other. Therefore, the multi-section bending tube having graduated rigidity of the present invention forms a front end with good flexibility, and the snake bone catheter portion 3 can be bent into a closed circular ring shape. After being bent, the deflection angle between the axis of a top end face of the snake bone catheter portion and the axis of the snake bone catheter portion before bending can exceed 270 degrees. Specifically, the rotation coaxiality of the straight catheter portion 1 is 100%, that is, the rotation axis of the straight catheter portion is always straight, and when angular deflection occurs at one end, the same angular deflection also occurs at the other end; the helical slits 21 are disposed on the catheter wall of the transition catheter portion 2, the helical slits 21 can provide a space for the bending of the tube body, so that the transition catheter portion can be bent, when the angular deflection occurs at one end, the same angular deflection also occurs at the other end, since the bending space provided by the slits 21 of the transition catheter section 2 is limited, the rotation coaxiality of this part of catheter is slightly lower than that of the straight catheter section 1, and the widths of the slits can be designed to meet the actual needs; the snake bone catheter portion 3 connected in series to the insertion end in front of the transition catheter section 2 includes a plurality of tube joints 31 connected with each other in series, the tube joints 31 are connected with each other in the clamping or hinging manner, and the snake bone catheter portion composed of the plurality of tube joints that are connected with each other in series can have greater twistability. When the straight catheter portion 1, the transition catheter portion 2 and the snake bone catheter portion 3 are sequentially connected, the rigidity of the multi-section bending tube 100 having graduated rigidity in the present embodiment gradually decreases from the non-insertion end to the insertion end, the twistability is gradually enhanced, compared with the traditional endoscope bending tube of a single structure, the multi-section bending tube having graduated rigidity has better controllability, can not only bend forward according to the internal cavity of the human body, but also can make the coaxial ratio of the insertion end to the non-insertion end maximally close to 1:1, and greatly reduce the coaxial error of the endoscope insertion tube, therefore the autogenous twisting of the laser optical fiber can be reduced, the service life of the instrument is prolonged, furthermore, the friction coefficient between the endoscope body and the ureter sheath is reduced, the operation difficulty is reduced, and the practical value is very high.

Preferably, as shown in FIG. 1, any tube joint 31 is provided with a first convex portion 32a that can be clamped in a corresponding first concave portion 33a of the adjacent tube joint, the first convex portion 32a and the first concave portion 33a both extend along the axial direction of the tube body, the first convex portion 32a and the first concave portion 33a cooperate with each other, the width of the front end of the first convex portion 32a is greater than the width of the tail end thereof (as shown by the arrow direction in FIG. 1, A represents the front end, and B represents the tail end), correspondingly, the width of the front end of the first concave portion 33a is greater than the width of the tail end thereof, so that the first convex portion 32a and the first concave portion 33a are clamped with each other. When the controllable bending tube is stretched along the axial direction, since the width of the front end of the first convex portion 32a is greater, the first convex portion cannot pass through the narrower rear end of the first concave portion 33a, so that the respective tube joints 31 are clamped in the axial direction without being excessively stretched, and at the same time, the excessive twisting of the multi-section bending tube having graduated rigidity in the present invention in the radial direction is further avoided.

Preferably, in the present embodiment, the length of the straight catheter portion can be 5.5% to 6% of that of the multi-section bending tube having graduated rigidity, the length of the transition catheter portion is 82% to 83.5% of that of the multi-section bending tube having graduated rigidity, the length of the snake bone catheter portion is 11% to 12% of that of the multi-section bending tube having graduated rigidity, wherein the length of the grouped tube joint portion is approximately 45% of the length of the snake bone catheter portion. The multi-section bending tube having graduated rigidity meeting the above-mentioned length ratio range can obtain the optimal rotation coaxiality. Those skilled in the art can think of that the length ratios of various parts can also be designed according to the actual needs.

More preferably, as shown in FIG. 4, one or more convex ribs 4 are formed on an inner wall of the multi-section bending tube having graduated rigidity, a traction hole 41 is further formed in each convex rib 4, the traction hole 41 can be used for accommodating a traction wire 42, and the bending of the multi-section bending tube having graduated rigidity can be controlled by controlling the traction wire 42. One or more convex ribs 4 extending from one end of the tube to the other end of the tube are formed on the inner tube wall of the multi-section bending tube having graduated rigidity, the traction hole 41 is further formed in each convex rib 4, the shape of the traction hole 41 is not particularly limited, as long as the traction hole 41 can be used for accommodating the traction wire 42, for example, the traction hole can be circular or oval, and the bending of the multi-section bending tube having graduated rigidity can be controlled by controlling the traction wire 42. When the multi-section bending tube having graduated rigidity disclosed in the present invention is manufactured, a tube with the convex rib 4 and the traction hole 41 in the convex rib 4 can be directly prepared by an extrusion die and other methods, and the tube with the convex rib 4 and the traction hole 41 is integrally formed directly. Thereafter, the tube becomes a bendable tube through common cutting methods in the industry.

Further, the number of the convex ribs 4 is two, and the tube can be controlled to bend in two directions when the traction wire 42 is inserted; and more preferably, the number of the convex ribs 4 is four, and the tube can be bent toward multiple angles through the cooperation of traction forces in multiple directions.

Further, the center line of the tube is taken as an axis in the multi-section bending tube having graduated rigidity, and preferably, the convex ribs 4 are symmetrically disposed in the controllable bending tube. The number of the convex ribs 4 corresponds to the number of the traction wires 42 inserted therein. When there are two convex ribs 4, that is, two traction holes 41 are formed for enabling the traction wires 42 to pass through, two corresponding traction wire 42 are also provided, the insertion end of the endoscope can be controlled to bend in two directions where the traction wires 42 are located, and it can be ensured that various angles are observed by rotating the endoscope insertion tube 100; and when there are four convex ribs 4, the insertion end of the endoscope can be controlled to bend in any direction, and any angle in the cavity can also be observed without rotating the endoscope.

Further, the multi-section bending tube having graduated rigidity is made of a metal material or plastic. The non-metal material can be selected from various plastic materials such as nylon, polyurethane (PU), thermoplastic polyurethane elastomer rubber (TPU), polyvinyl chloride (PVC), thermoplastic elastomer (TPE), etc.

Further, in the present preferred embodiment, the traction wire 42 is preferably a steel wire, but the present invention is not limited thereto, the traction wire 42 can also be any filament-like structure, and the filament-like structure can also be selected from any metal or non-metal material.

Further, the protective sheath is preferably made of polyvinyl chloride, polyurethane, block polyetheramide elastomer or polyamide. The polyvinyl chloride material has common biocompatibility, slightly better strength, but has relatively high hardness and worse bendability, and thus cannot be bent according to the cavity demands under a human body temperature; the polyamide has better biocompatibility, but has greater hydroscopicity, so that the size stability is affected to a certain extent in the complicated internal environment of the human body, and the biocompatibility and strength thereof are slightly worse than those of polyurethane. However, these two materials are low in cost and widely used in industry, and can also meet the use needs of the insertion end of the endoscope.

In summary, according to the multi-section bending tube having graduated rigidity, the insertion tube for the endoscope using the bending tube, and the endoscope involved in the present invention, the multi-section bending tube having graduated rigidity is formed by sequentially connecting three-section catheters with different structures, compared with the traditional endoscope insertion tube, the coaxial steering proportion of the endoscope insertion tube can be improved, so that the axial ratio of the insertion end to the non-insertion end is maximally close to 1:1, and the coaxial error of the endoscope insertion tube is greatly reduced, so that the autogenous twisting of the laser optical fiber can be reduced, the service life of the instrument is prolonged, furthermore, the friction coefficient between the endoscope body and a ureter sheath is reduced, the operation difficulty is reduced, and the practical value is very high.

In addition, the above embodiments merely illustrate the principle of the present invention and its effects, but are not intended to limit the present invention. Any skilled in the art can modify or change the above embodiments without departing from the spirit and scope of the present invention. Therefore, all equivalent modifications or changes made by those skilled in the art without departing from the spirit and technical ideas disclosed by the present invention should still be encompassed by the claims of the present invention.

1. A multi-section bending tube having graduated rigidity, applied to an insertion portion of an endoscope, wherein the multi-section bending tube having graduated rigidity sequentially comprises, from a non-insertion end to an insertion end: a straight catheter portion, a transition catheter portion and a snake bone catheter portion, which have equal inside diameters and are connected in sequence, the straight catheter portion is a cylindrical catheter with a fully-enclosed catheter wall, slits for providing a bending space of a tube body are disposed on the catheter wall of the transition catheter portion, the snake bone catheter portion comprises a plurality of tube joints connected with each other in series, the tube joints are connected with each other in a clamping or hinging manner, and the curvature of the transition catheter portion is greater than the curvature of the straight catheter portion and is less than the curvature of the snake bone catheter portion.

2. The multi-section bending tube having graduated rigidity according to claim 1, wherein helical slits are disposed on the catheter wall of the transition catheter portion, and the screw pitches among the helical slits of the transition catheter portion are equal.

3. The multi-section bending tube having graduated rigidity according to claim 2, wherein the transition catheter portion and the snake bone catheter portion are connected by an elongated tube joint.

4. The multi-section bending tube having graduated rigidity according to claim 2, wherein the snake bone catheter portion comprises a grouped tube joint portion and an isometric tube joint portion from the non-insertion end to the insertion end, the grouped tube joint portion comprises multiple groups of snake bone catheters, and each group of snake bone catheters is composed of a plurality of tube joints with the same length and connected in series, the groups of snake bone catheters are connected by elongated tube joints, and the numbers of tube joints of the groups of snake bone catheters gradually increase from the non-insertion end to the insertion end.

5. The multi-section bending tube having graduated rigidity according to claim 1, wherein the axial lengths of the plurality of tube joints of the snake bone catheter portion gradually decrease from the non-insertion end to the insertion end.

6. The multi-section bending tube having graduated rigidity according to claim 1, wherein any tube joint is provided with a convex portion that can be clamped in a corresponding concave portion of the adjacent tube joint.

7. The multi-section bending tube having graduated rigidity according to claim 6, wherein a seam with a certain width is disposed at the connection site of the tube joints on both sides of the convex portion of the snake bone catheter portion.

8. The multi-section bending tube having graduated rigidity according to claim 1, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

9. An insertion tube for an endoscope, comprising:
the multi-section bending tube having graduated rigidity according to claim 1; and a protective sheath covering the multi-section bending tube having graduated rigidity.

10. An endoscope, comprising:
the insertion tube for the endoscope according to claim 9, a control device and a handle; one or more traction wires are disposed in the insertion tube, one end of the traction wire is fixedly connected with the insertion end of the insertion tube, and the other end of the traction wire is connected with the control device disposed on the handle for controlling the bending of the insertion tube.

11. The multi-section bending tube having graduated rigidity according to claim 2, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

12. The multi-section bending tube having graduated rigidity according to claim 3, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

13. The multi-section bending tube having graduated rigidity according to claim 4, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

14. The multi-section bending tube having graduated rigidity according to claim 5, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

15. The multi-section bending tube having graduated rigidity according to claim 6, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

16. The multi-section bending tube having graduated rigidity according to claim 7, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.

17. The insertion tube for an endoscope according to claim 9, wherein helical slits are disposed on the catheter wall of the transition catheter portion, and the screw pitches among the helical slits of the transition catheter portion are equal.

18. The insertion tube for an endoscope according to claim 16, wherein the transition catheter portion and the snake bone catheter portion are connected by an elongated tube joint.

19. The insertion tube for an endoscope according to claim 16, wherein the snake bone catheter portion comprises a grouped tube joint portion and an isometric tube joint portion from the non-insertion end to the insertion end, the grouped tube joint portion comprises multiple groups of snake bone catheters, and each group of snake bone catheters is composed of a plurality of tube joints with the same length and connected in series, the groups of snake bone catheters are connected by elongated tube joints, and the numbers of tube joints of the groups of snake bone catheters gradually increase from the non-insertion end to the insertion end.

20. The insertion tube for an endoscope according to claim 9, wherein the axial lengths of the plurality of tube joints of the snake bone catheter portion gradually decrease from the non-insertion end to the insertion end.

21. The insertion tube for an endoscope according to claim 9, wherein any tube joint is provided with a convex portion that can be clamped in a corresponding concave portion of the adjacent tube joint.

22. The insertion tube for an endoscope according to claim 20, wherein a seam with a certain width is disposed at the connection site of the tube joints on both sides of the convex portion of the snake bone catheter portion.

23. The insertion tube for an endoscope according to claim 9, wherein one or more convex ribs are formed on an inner wall of the multi-section bending tube having graduated rigidity, a traction hole is further formed in each convex rib, the traction hole can be used for accommodating a traction wire, and the bending of the multi-section bending tube having graduated rigidity can be controlled by controlling the traction wire.
